(19) United States
(12) Patent Application Publication
(10) Pub. No.: US 2023/0050627 A1
(43) Pub. Date: Feb. 16, 2023

(54) SYSTEM AND METHOD FOR LEARNING TO GENERATE CHEMICAL COMPOUNDS WITH DESIRED PROPERTIES

(71) Applicant: 99ANDBEYOND INC., Montreal (CA)

(72) Inventors: Boris SATTAROV, Saratov (RU); Vijaya Sai Krishna GOTTIPATI, Waterloo (CA); Yashaswi PATHAK, Patna (IN); Karam THOMAS, Saint-Laurent (CA)

(73) Assignee: 99ANDBEYOND INC., Montreal, QC (CA)

(21) Appl. No.: 17/796,826

(22) PCT Filed: Jan. 29, 2021
(86) PCT No.: PCT/CA2021/050103

§ 371 Date: Aug. 1, 2022

Related U.S. Application Data
(60) Provisional application No. 62/967,898, filed on Jan. 30, 2020.
(60) Provisional application No. 63/076,151, filed on Sep. 9, 2020.

(51) Int. Cl. G16C 20/50; G06F 30/27; G16C 20/70; G16C 20/10

(52) U.S. Cl. CPC G16C 20/50; G06F 30/27; G16C 20/70; G16C 20/10

(57) ABSTRACT

A system and method for generating libraries of chemical compounds having desired and specific properties by formulating a reaction-based mechanism that may be powered by several algorithms including but not limited to genetic algorithm, expert iteration algorithms, planning methods, reinforcement learning and machine learning algorithms. The system and method may also provide the process steps by which these optimized products S′ may be synthesized from the reactants R1,R2 and further enables a rapid and efficient search of the synthetically accessible chemical space.

### CROSS-REFERENCE TO RELATED APPLICATIONS

This application claims priority from U.S. Provisional Patent Application No. 62/967,898, filed on Jan. 30, 2020, entitled “SYSTEM AND METHOD FOR LEARNING TO GENERATE CHEMICAL COMPOUNDS WITH DESIRED PROPERTIES,” the entire contents of which are hereby incorporated by reference.

This application claims priority from U.S. Provisional Patent Application No. 63/076,151, filed on Sep. 9, 2020, entitled “SYSTEM AND METHOD FOR LEARNING TO GENERATE CHEMICAL COMPOUNDS WITH DESIRED PROPERTIES,” the entire contents of which are hereby incorporated by reference.

### TECHNICAL FIELD

The present invention pertains to the fields of chemistry and algorithmic or machine learning chemical reaction prediction for the generation of novel pharmaceutical drugs, materials, cosmetics, pesticides, or other chemical compounds.

### BACKGROUND

Strategies for conducting machine learning-based de novo drug design can be divided into two groups: structure generating schemes and reaction-based schemes.

Structure generating schemes are machine learning models which are trained to produce chemical compounds without any explicit notion of the chemical reactions which can be used to synthesize them. Structure generating schemes may utilize encoder/decoder-based generative systems as well as reinforcement learning systems. However, these structure generating schemes may often lead to molecules which are impossible or infeasible to manufacture. To compensate, a synthetic accessibility/feasibility score is usually accounted for through the introduction of data-driven estimation into the schemes' scoring function module. Retrosynthesis analysis is also typically conducted after a final chemical compound is generated.

Structure generating schemes have two significant disadvantages. First, generative models frequently tend to exploit data-driven artifacts in the discriminator model, which can guide generation in the wrong direction and compromise real-world synthetic accessibility of the generated structures. Second, any data-driven model that predicts synthetic accessibility/feasibility score will have limited applicability depending on the training set that was used to train the model. These disadvantages lead to greater computation time and a higher generation of results with little utility.

These disadvantages may be overcome through the use of reaction-based models for the generation of novel chemical compounds. By basing an algorithm's exploration of a chemical space on known reactions and commercially or synthetically available reactants, the efficacy of the scoring function module can be increased, and the overall productivity and efficiency of the generation scheme may be enhanced.

However, existing reaction-based models present challenges. Examples such as DINGOS or PathFinder are limited in two major ways. First, both systems require a known template lead compound for the biological target of interest in order to be applicable. Second, these systems are not trained in an end-to-end manner. In the case of PathFinder, for example, compounds are first generated using reactions and only then some products are selected using a disconnected scoring function module. In the case of DINGOS, only the part that predicts the possible second reactant is actually trained, and this training is only conducted in a supervised manner using reaction data.

The method and process described herein overcomes these limitations, enabling an end-to-end reaction-based search of the synthetically accessible chemical space by using a set of available reactants. One or more template compounds are no longer required.

### SUMMARY

A system and method for generating libraries of chemical compounds are described herein. The system and method utilize a reaction-based scheme that is guided by machine learning, including but not limited to reinforcement learning or expert iteration algorithms, genetic algorithms, and/or planning methods, and comprises a scoring function module for the purpose of generating chemical candidates which exhibit desired properties, characteristics and/or behaviors. Through this process, the system and method also generate and display the corresponding methods by which these chemical candidates may be synthesized and/or manufactured. Thus, it becomes possible to efficiently search the vast majority of the synthetically accessible chemical space in a relatively short time frame.

### BRIEF DESCRIPTION OF THE FIGURES

Advantages of embodiments of the present invention will be apparent from the following detailed description of the exemplary embodiments thereof, which description should be considered in conjunction with the accompanying drawings in which like numerals indicate like elements, in which:

FIG. 1 is an exemplary embodiment of a reinforcement learning workflow.

FIG. 2 is an exemplary embodiment of an actor module of a reinforcement learning workflow.

FIG. 3 is an exemplary embodiment of a critic module of a reinforcement learning workflow.

FIG. 4 is an exemplary embodiment of an environment of a reinforcement learning workflow.

FIG. 5 is an exemplary embodiment of a reinforcement learning workflow which has a double actor and critic workflow.

FIG. 6 is an exemplary embodiment of a reinforcement learning workflow which has a multiple actor and critic workflow.

FIG. 7 is an exemplary embodiment of a reinforcement learning workflow with multiple actors and critics.

FIG. 8 is an exemplary embodiment of a reinforcement learning workflow utilizing a differentiable k-nearest neighbors module.

FIG. 9 is an exemplary embodiment of a reinforcement learning workflow that learns to choose the initial reactant(s).

FIG. 10 is an exemplary embodiment of a genetic algorithm workflow.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more classical or quantum processors, or by a combination of both. Additionally, these sequences of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

Reinforcement learning is a paradigm of machine learning in which an algorithm seeks to map out an environment and make decisions in order to maximize an overall reward metric. One way of implementing a reinforcement learning scheme is through a Markov Decision Process (MDP). The Markov Decision Process is a mathematical framework for describing an underlying reinforcement learning task. This mathematical framework is characterized by a state S, action A, transition function P, reward M, and optionally discount factor gamma at every time step. The transition function P represents the probability that an action A in state S at any time step t will lead to state S′ at time step t+1. The goal of a Markov Decision Process framework is to find a policy for an agent which will maximize some function of the rewards M at each time step. The rewards M may optionally be scaled by a discount factor γ.

The Markov Decision Process is preferably structured as an end-to-end reinforcement learning workflow. This end-to-end structure allows for the reinforcement learning workflow to learn not just actions pertaining to a specific task, but to learn the entire process up to and including higher-level functions that may be difficult to develop independently from other functions. This allows for a more sophisticated agent to be trained and for a broader array of reactions and if necessary corresponding conditions to be considered.

Reinforcement learning algorithms may be classified into many different types: model-based, model-free, on-policy, and off-policy. These algorithms may also be classified based on an update rule, such as value-based or policy gradient methods. Policy gradient methods may deal with discrete action spaces or continuous action spaces. Examples of discrete action space algorithms include REINFORCE, actor-critic, advantage actor-critic, trust region policy optimization, ACKTR, and proximal policy optimization.

However, because the discrete action space is very large in the case of chemical compound generation, algorithms are preferred which are adapted to or correspond to a continuous action space. Examples of algorithms which operate in a continuous action space include deterministic policy gradient, deep deterministic policy gradient (DDPG), distributed distributional deep deterministic policy gradient (D4PG), twin delayed deep deterministic policy gradient (TD3), and soft actor critic (SAC).

Another method according to the present invention for managing a large, discrete action space involves predicting an action in the continuous space and then using a k-nearest neighbors (kNN) algorithm to map the continuous space action to one or more valid discrete actions. In order to accomplish this, a distance metric is introduced in which neighbors that are “closer” to a given input contribute more than those neighbors that are “further” from the input. Any distance or divergence metric may be used to evaluate the “close-ness” metric. Algorithms with properties similar to k-nearest neighbor may also be used.

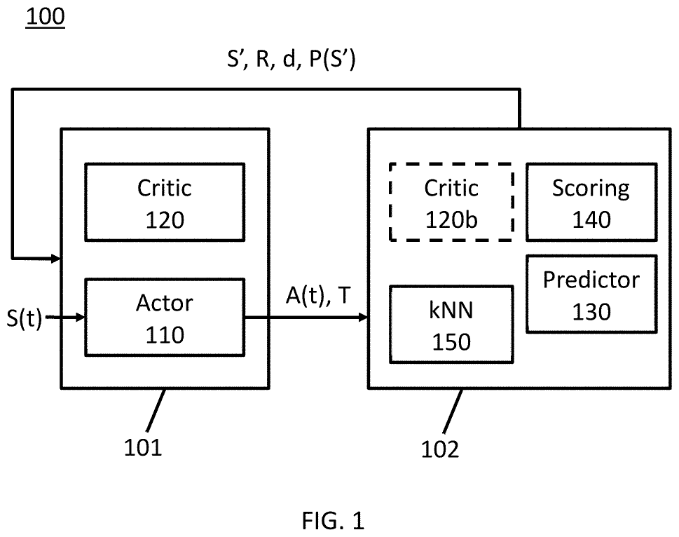

Turning now to FIG. 1, an exemplary embodiment of an MDP workflow 100 is displayed, featuring an agent 101 with at least one actor module 110 and at least one critic module 120. Agent 101 interacts with environment 102, which is described by reaction predictor 130 mapped into a discrete space by a kNN algorithm 150 and scored according to a scoring function 140. Environment 102 may also optionally contain a copy critic module 120b which replicates critic module 120 as described above.

At every timestep t, a reactant R2(t) reacts with an existing molecule or reactant R1(t) to produce a product R1(t+1). R1(t) may also be represented by the state S(t), R2(t) may also be represented by the action A(t), and the product R1(t+1) may be represented as the state S′(t+1) for the following timestep.

At an initial timestep, the initial molecule R1(t=0) is sampled from a list of all available reactants. This sampling may be random, statistically driven, selected based on a scoring function module, or selected according to a neural network module trained in an end-to-end manner similar to that described herein. Because the potential action space is very large, it is preferable to introduce an intermediate action A1(t) which may reduce the size of the action space. Intermediate action A1(t) may take the form of a reaction that serves as a filter for the action space. This reaction preferably filters the action space on the basis of the active sites of one or both of the reactants R1(t) and R2(t) as a metric. The reaction preferably filters the action A(t) and/or the reactant R2(t). It may also be preferable to conduct further filtering for any of the reactants and reactions.

Reactants may be encoded in a variety of formats. If the reactant R is encoded in a domain-specific vector representation of molecular structures, then it may be directly passed through the relevant networks. If, however, the reactant R is encoded in a graph format, it may be passed through a learnable or pre-trained graph convolution or any other type of layer to obtain a compact representation of the reactant. If a reactant R is encoded in another different, but likewise incompatible format, the reactant is passed through an appropriate learnable layer to be converted to an appropriate, compatible, and compact representation. This compact representation may be the same as the desired domain-specific vector representation or may also be a functional equivalent thereof.

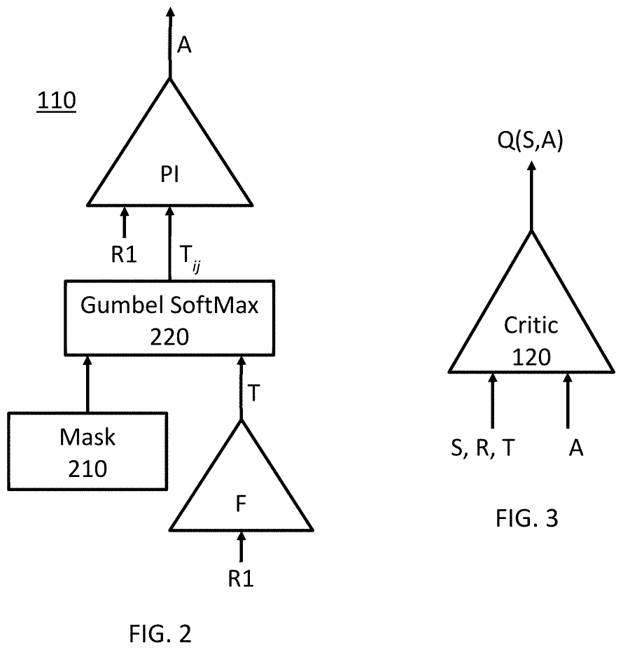

Turning now to FIG. 2, an exemplary embodiment of an actor module 110 of the reinforcement learning workflow is displayed. Two learnable networks, F and PI, may be used in the network constituting the actor module 110, though any number of neural network layers of any type and with any activation unit may be used. Any learnable modules that have learnable parameters may be used.

Optionally, the output of the F network may be multiplied element-wise with a template mask 210. This template mask 210 is a binary vector or tensor in which values of 1 represent a valid template and values of zero represent an invalid template for a given reactant. The output of this multiplication may then be passed through a Gumbel-Softmax layer 220 to obtain a one-hot vector/tensor Tᵢⱼ representing the best reaction T. Reactant R1(t) along with this reaction T are then used as inputs to the PI network to compute a proto action. This proto action may thus have reactant R2 in a continuous space, which is typically the space defined by embeddings of all actions A.

Turning now to FIG. 3, an exemplary critic module 120 of the reinforcement learning workflow is depicted. In the context of a reinforcement learning framework, the critic module 120 evaluates the output of the actor module. Inputs to the critic module 120 are typically the state S(t), reactant R1(t), and/or the reaction T(t) as well as the action A(t). Action A(t) may be input to the critic module as the proto action and/or the reactant R2(t). The goal of the critic module 120 is to calculate or evaluate the “goodness” Q(S,A) of the action. Workflows utilizing more than one actor and/or critic modules 110, 120 may be possible.

Optionally, the critic module 120 may also be used to choose the best reactant or reactants R among choices presented by the output of a k-nearest neighbor module 150. These choices may be drawn from all of the valid reactants dictated by the reaction, with or without consideration of R1(t).

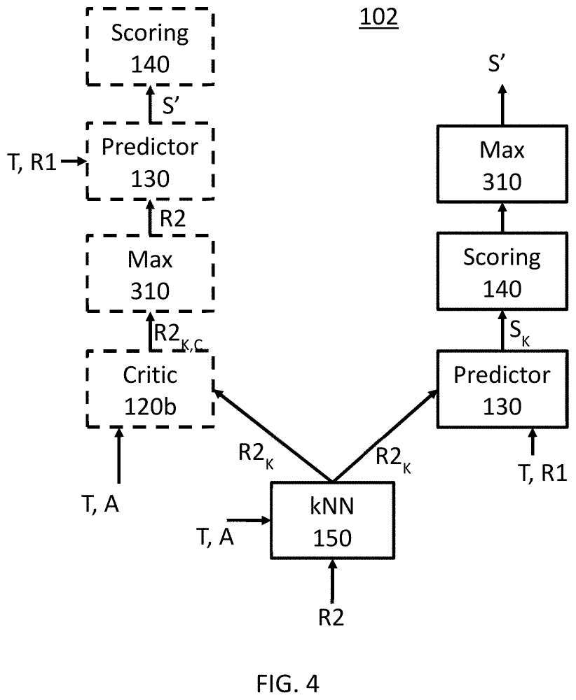

Turning now to FIG. 4, an exemplary, detailed workflow of the environment 102 used in the reinforcement learning workflow is described. The environment 102 takes in the proto action, the best reaction T, and/or the current action A. The environment 102 then predicts the next state(s) S(t+1), corresponding reward(s) for the next state(s) S(t+1), termination of the episode, and/or probabilities of each of the next state(s) S(t+1) if applicable. The environment 102 also includes the k-nearest neighbor module 150, a reaction predictor 130, a scoring function module 140, a maximum and/or arg-maximum operator 310, and/or a copy of the agent's critic module 120b.

During this process, the environment 102 passes the proto action, best reaction T, and/or the vector/tensor representations of the rest of all reactants R2(t) as inputs into the k-nearest neighbor module 150 to obtain the k-nearest neighbors to the proto action that fits the best reaction T of all reactants R2(t). These k-valid reactants R2_{K}(t) are then passed along with reactant R1(t) and the best reaction T through the reaction predictor 130 module to obtain corresponding k products S_{K}(t+1), which are then evaluated by the scoring function module 140 to obtain the corresponding k rewards. The product corresponding to the maximum reward, as determined by the max and/or arg-max operators 310, is then chosen.

Optionally, the critic module 120 provided to the environment 102 may be used to evaluate the goodness Q(S,A) values of all the k valid reactants R2(t) and choose the reactant R2(t) corresponding to the best goodness value Q(S,A) for the given state S, as reactant R1(t) or best reaction T, and action A(t) or reactant R2(t) selected from the provided k valid reactants R2(t). This best reactant R2(t) along with reactant R1(t) and best reaction T are then passed through the reaction predictor module 130 to obtain the product(s) and/or the corresponding probabilities of each product. The obtained product(s) are then used as input to the scoring function module 140 which then computes the reward.

Optionally, the PI network output may be passed through the differentiable k-nearest neighbors module 150. The critic network 120 may then be used to select the best second reactant R2(t) from the k chosen reactants. The environment may, using its scoring function module 140, calculate the reward associated with the best second reactant.

The scoring function module 140 of the environment 102 may function according to a rule-based and/or physics-based method. The scoring functions 140 may also utilize machine learning-based methods. The goal of the scoring function module is to predict and/or determine the physical, chemical, functional, electrical, quantum mechanical, structural, biophysical and/or biochemical properties of the compounds involved in the reactions. The biochemical properties may, for example, describe activity on a single or multiple biological targets, such as receptors, enzymes, etc., associated with cells, tissues, or even entire organisms.

The reaction prediction modules 130 may be utilized to predict the outcome of a chemical reaction based upon the provided reactants and reaction and if necessary corresponding conditions. The prediction models 130 may also leverage N-component transformations, which represent a type of reaction where a second reactant is unnecessary; only a single reactant, reaction and if necessary corresponding conditions are necessary. The prediction module may be structured to accommodate reaction using SMARTS or other formats and representations.

The reaction prediction module 130 of the environment 102 is also provided to utilize the aforementioned methods to determine the end of a single or multi-step virtual synthesis route which may thus constitute an episode.

An “episode” is defined as the process constituting synthesis of an ultimate or intermediate product. Episodes are composed of at least one step, the step including information relevant to carrying out that step of the process. A step may for example, include the reactants used, any environmental factors which may be necessary to facilitate the reaction, and/or any catalysts or other non-reactive components necessary for achieving. In this manner, an episode is meant to provide to a user a sort of recipe or means by which a final or intermediate product may be synthesized.

The reward and/or scoring function module 140 of the environment 102 score the reactants and/or products according to the predicted and/or experimental physical, chemical, functional, electrical, quantum mechanical, structural, biophysical and/or biochemical properties relative to desired and/or specific properties.

These chemical properties, along with the reactants and/or products, may be stored in a machine-readable format. This machine-readable format may be, at various stages, converted between human-readable formats and those formats preferable to the machine learning workflow.

The scoring function module 140 may take a chemical compound as an input and, in turn, outputs a corresponding value associated with one or more properties, behaviors and/or characteristics of the compound. The scoring function module 140 can but is not limited to utilizing a machine learning model and/or ensemble of models, a molecular or quantum mechanics simulation, and/or experimental values. The scoring function module 140 may combine one or more of these properties with one or more of these methods by utilizing weighting factors.

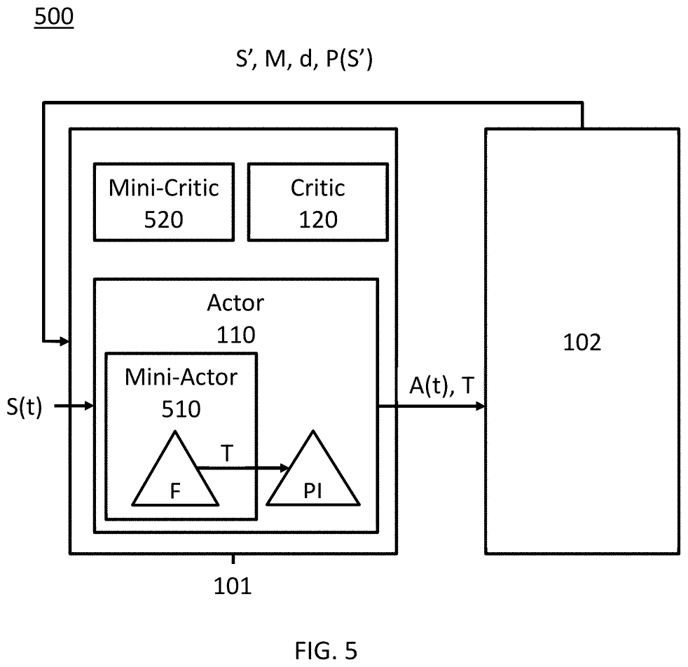

Turning to FIG. 5, an exemplary workflow describing a double actor-critic workflow 500 as part of the reinforcement learning workflow is shown. The reinforcement learning workflow may contain a double actor and/or critic workflow 500 instead of a single actor-critic workflow. In this manner, a mini-actor 510 and a mini-critic 520 accompany their actor-critic counterparts 110, 120. Workflows containing more than one mini-actor 510 modules and/or mini-critic 520 modules may also be possible. Workflows containing only one or more mini-actor modules 510 in addition to the actor-critic modules 110, 120, or workflows containing only one or more mini-critic modules 520 in addition to the actor-critic modules 110, 120 may be possible.

The mini-actor module 510 may output a vector/tensor indicating probabilities of reaction to be chosen given at least one reactant as input. The mini-critic module may, if necessary, evaluate the output of the mini-actor module.

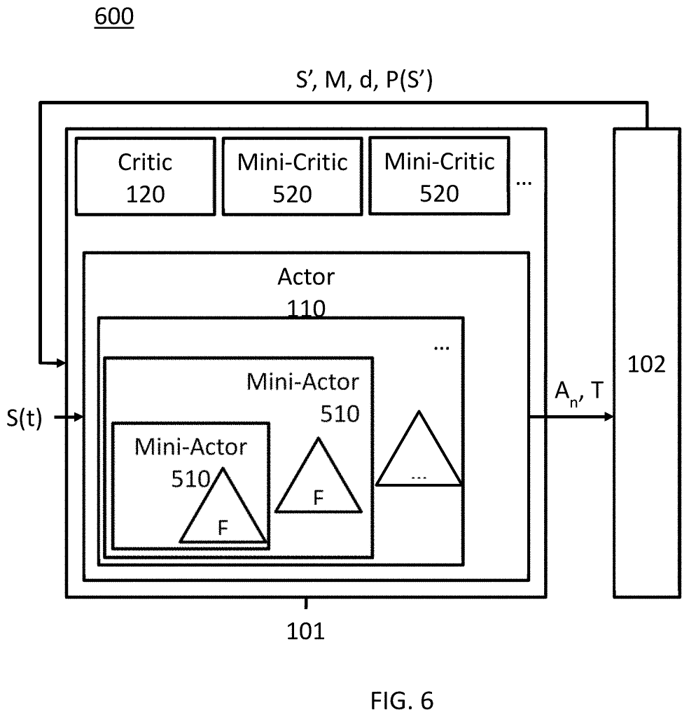

Another embodiment of the double actor-critic workflow 500, a multiple actor-critic workflow 600, is illustrated in FIG. 6. As with the double actor-critic workflow 500, the multiple actor-critic workflow 600 may utilize any number of actor 110, critic 120, mini-actor 510, or mini-critic modules 520 as necessary.

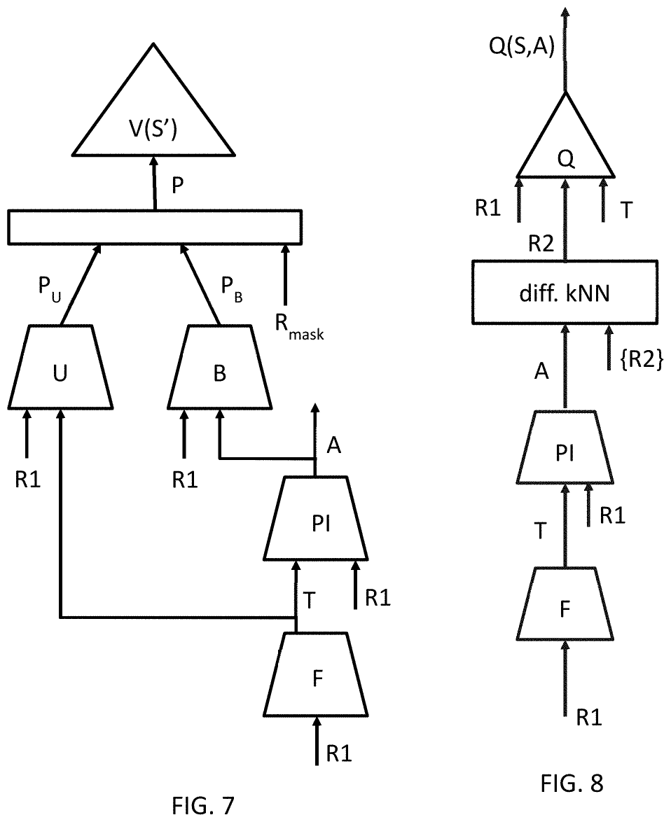

Another embodiment of the double actor-critic workflow 500, synonymously described as a pyramidal actor-critic workflow, may be formulated as follows and is depicted in FIG. 7. Under the assumption of a deterministic transition function, the value function of the next state V(s′) is exactly equal to Q(S,A) value of the current state S and action A pair. This assumption allows the “critic” to be broken down into a two-step process, defined by two modules internal to the critic: a product predictor and a value function predictor. The value function predictor predicts, for example, the value function V(s′) of the next state s′ of the product. The product predictor has two different networks, a U-net for processing uni-molecular reactions and a B-net for processing bi-molecular reactions.

U-net takes in R(1) and reaction T, or any representations thereof, as inputs and computes a representation of a hypothetical product according to: Pᵤ=U_{θ}_{_{U}}(R⁽¹⁾),T)

B-net takes R(1), R(2) and reaction T, or any representations thereof, as inputs and computes a representation of a hypothetical product according to: P_{b}=B_{θ}_{_{B}}(R⁽¹⁾,R⁽²⁾,T)

The two hypothetical products are then combined to compute a hypothetical final product P of the chemical reaction using an appropriate Rₘₐₛₖ depending on whether or not the reaction is uni-molecular or bi-molecular according to: P=Pᵤ*(1−Rₘₐₛₖ)+P_{b}*Rₘₐₛₖ

This final hypothetical product obtained from these hypothetical product predictor modules is passed through the learnable value function module V to obtain Q(S,A).

Another embodiment of a pyramidal actor-critic workflow may be formulated as follows and is depicted in FIG. 8. Consider a policy network PI with L layers, where θ_{L} denotes parameters of the PI network, as well as the various sub-networks within the PI network where PI_{θ}_{ₗ} denotes layers 0 to l within the policy network PI. These layers constitute one of the L such possible mini-policy networks. Next, consider another neural network Cₗ that takes in the current state and the output of layer l of the policy model PI. This may also be seen as output of the PI_{θ}_{ₗ} network. The neural network Cₗ predicts the hypothetical next state. The output of Cₗ need not be in the space of next states—it may be any representation space.

One such hypothetical next state prediction module (HyNeSP) can be assigned to each of the mini-policy modules PI_{θ}_{ₗ}. These states are only hypothetical because one may not predict the true next state without the actual action A, which may be the output of PI_{θ}_{ₗ}.

Now, considering the environment with a deterministic transition function, which is to say, only one next state s′ is possible given current state S and action A. In such cases, the Q(S,A) function of current state S and action A is equal to the value function of the next state S′, or: Q(S,A)=V (S′)

The algorithms here may still be used even if the transition function is non-deterministic, or when: Q(S,A)≠V(S′)

Once a hypothetical next state h′ has been obtained using one of the HyNeSPs, computing Q(S,A) becomes equivalent to computing the value function of the hypothetical next state. Thus, a new network V may be introduced that takes as input the hypothetical next state h′ and predicts its value function.

The hypothetical product to be chosen from m different HyNeSPs is determined by sampling from a fixed or learnable probability tensor and converting it to a one-hot tensor, leading to HyNeSP mask Mₕ.

Another method for training involves utilizing cross-entropy methods or, more broadly, model predictive control. Noise may be added to the outputs of the actor and, based on the rewards, an optimal noise distribution may be determined and/or computed. This noise distribution may be initially modeled using any probability distribution. This process may be used on pre-trained actor networks and/or during the training phase. Optionally, the noise may be directly added to the parameters of the networks.

Another method for training the actor networks may utilize supervised learning via an expert agent, like-expert demonstrations and/or Monte Carlo Tree Search (MCTS) simulations. Novel training strategies for dealing with MCTS in continuous action spaces may be introduced. The policy loss or the actor loss aims to minimize the divergence between the output policy distribution and the target policy distribution.

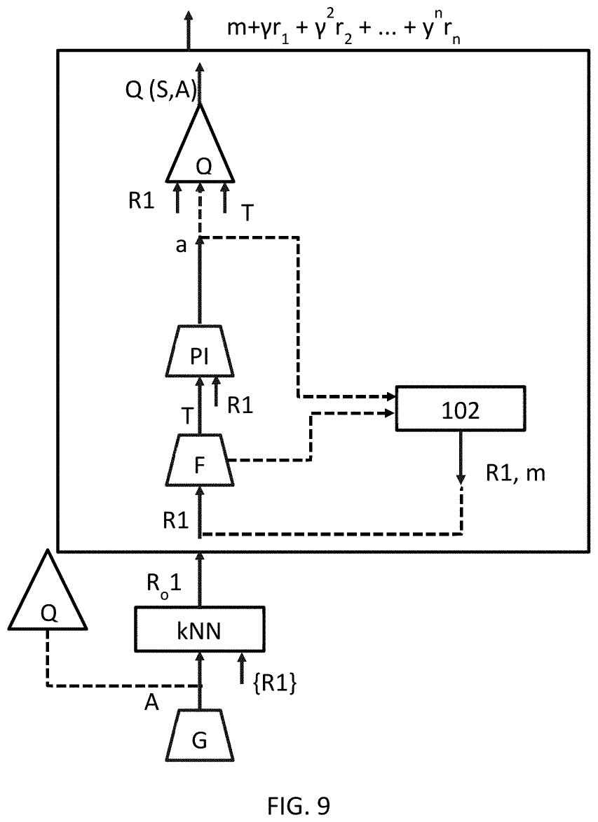

Turning to FIG. 9, a reinforcement learning workflow which learns to choose initial reactant(s) is depicted. One potential embodiment of the present invention includes introducing a new objective function to the existing reinforcement learning framework. While existing approaches are focused mostly on optimizing the overall reward, discounted or undiscounted, or a function of reward, discounted or undiscounted, with a varying number of time steps in a finite or infinite episode setting, the present invention may instead optimize the maximum reward achieved in an entire episode. The present invention may utilize a novel Bellman equation for Q-function and other functions/variables used in any reinforcement learning setting to optimize the new objective.

The Q-function may be defined as:

$${Q}_{m a x}^{π} ( {s}_{t} , {a}_{t} ) = {E}_{\begin{matrix}{s}_{t + 1 \sim P ( \cdot \vert {s}_{t} , {a}_{t} )} \\ {a}_{t + 1} \sim π ( \cdot \vert {s}_{t + 1} )\end{matrix}} [ max ( M ( s , a ) , γ {E}_{\begin{matrix}{s}_{t + 2} \sim P ( \cdot \vert {s}_{t + 1} , {a}_{t + 1} ) \\ {a}_{t + 2} \sim π ( \cdot \vert {s}_{t + 2} )\end{matrix}} [ max ( M ( {s}_{t + 1} , {a}_{t + 1} ) , … ) ] ) ]$$

And, correspondingly, the Bellman equation may take the form of:

$${Q}_{m a x}^{π} ( {s}_{t} , {a}_{t} ) = {E}_{\begin{matrix}{s}_{t + 1} \sim P ( \cdot \vert {s}_{t} , {a}_{t} ) \\ {a}_{t + 1} \sim π ( \cdot \vert {s}_{t + 1} )\end{matrix}} max [ M ( {s}_{t} , {a}_{t} ) , γ {Q}_{m a x}^{π} ( {s}_{t + 1} , {a}_{t + 1} ) ]$$

The return of an episode may be defined as:

$$M ( τ ) = {max {m}_{t}}_{t = 0}^{T} .$$

It can optionally be scaled by the discount factor gamma and the return may be defined as

$$M ( τ ) = {max {γ}^{t} m}_{t = 0}^{T} .$$

In contrast to existing methods where the initial state is fixed, given, or randomly sampled, the present invention may learn to choose an initial state. A random noise is sampled and may be passed through a generator network G whose output is in the space of any preferred representation of reactants. The output of the generator network G may then be mapped to a valid initial reactant using k-nearest neighbors algorithms. To promote the diversity of the generated molecules without compromising on the rewards achieved, a technique may be employed to avoid collapsing the generator network G to a single point or single region. Examples of techniques which may be used to this end include, but are not limited to: regularization, soft k-means clustering by maximizing inter-cluster distance of the outputs of the generator network, modifying rewards from the environment to reward diversity, or using multiple generators. Optionally, an additional critic network may be used to evaluate the output of the generator network G and update the parameters of the generator network G in an actor-critic fashion. Alternatively, a different policy gradient algorithm may be used instead of k-nearest neighbors, such as any differentiable version of k-nearest neighbors.

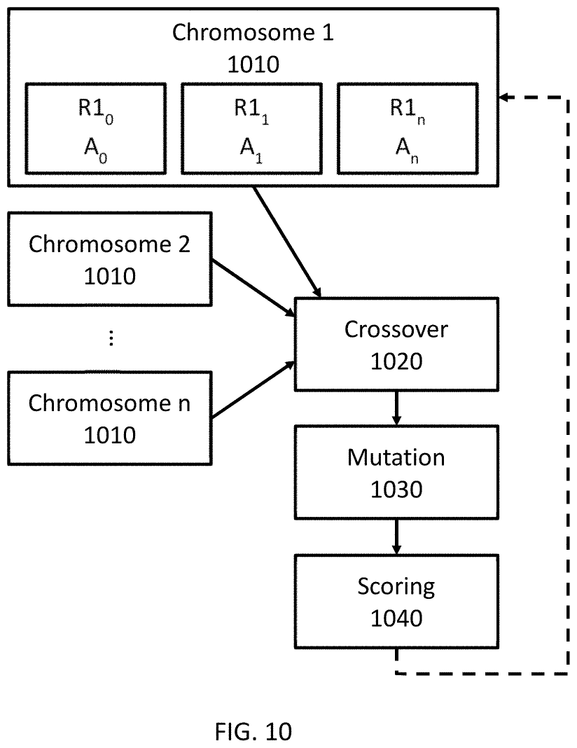

Another method for training the forward synthesis framework can utilize genetic algorithm agents as an alternative to reinforcement learning agents as depicted in FIG. 10. In genetic algorithms, a set of genes or features that an individual possesses is called a chromosome 1010. Each individual chromosome in the population can be represented as a sequence of proto actions in the space of the reactants similar to the reinforcement learning-based implementation. A first part of a chromosome starts with a multidimensional proto action in the space of the reactant's features which is responsible for the selection of the initial reactant R1. Optionally, this step may be skipped and any other method for choosing the initial reactant R1 may be used. Subsequent parts of a chromosome are a sequence of the proto actions responsible for selection of the second reactants with which the state molecule will react at each step of the forward synthesis.

Each individual chromosome is then evaluated by the environment. The environment takes the chromosome and applies the k-nearest neighbor algorithm to the encoded proto actions to select a valid initial reactant and subsequent second reactants which are closest to the proto action in the defined feature space. Then, the environment carries out multi-step forward synthesis to generate chemical compounds at each step and computes a reward value of each molecule. Optimized reward values achieved during the multi-step forward synthesis process are encoded in each individual's chromosome and returned as its fitness value.

In the initial population generation, individual chromosomes are randomly initialized. Once the initial population is evaluated, the initial population undergoes crossover 1020. During crossover, features are randomly exchanged between two individual chromosomes to produce a set of offsprings. Crossover is followed by mutations 1030 which are random modifications of the feature values in the chromosome of the single individual. Following mutation, the best individuals are selected according to their rewards determined through the scoring function module 1040 and used to form a new population or generation. Crossover and mutation events are triggered for certain individuals with a predefined probability.

Optionally, one or more neural networks can be trained on the samples or proto actions generated by the genetic algorithm. Further, the samples from these neural networks may be used as initial chromosomes so that further crossover and mutation operations may be performed. These two processes may run simultaneously, sequentially, synchronously, and/or asynchronously to improve each other.

Optionally, one or more neural networks, actor and/or critic networks, may be trained in a reinforcement learning framework by sampling from the samples, such as tuples of reactants, proto action, products, and rewards, as generated by the genetic algorithm. The samples from these neural networks may be used as initial chromosomes and further crossover and mutation operations may be performed on these chromosomes. These two processes may run simultaneously, sequentially, synchronously, and/or asynchronously to improve each other.

Optionally, one or more neural networks, actor and/or critic networks, may be trained in a reinforcement learning framework to mimic, imitate, or replicate any of the genetic algorithms or planning methods described above.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art (for example, features associated with certain configurations of the invention may instead be associated with any other configurations of the invention, as desired).

Therefore, the above-described embodiments should be regarded as illustrative rather than restrictive. Accordingly, it should be appreciated that variations to those embodiments can be made by those skilled in the art without departing from the scope of the invention as defined by the following claims.

1-20. (canceled)

21. A system for automated design of molecules, comprising:
an artificial intelligence environment comprising a chemical reaction prediction module and a scoring function module,
wherein the artificial intelligence environment predicts a set of probable reaction products based on at least one reaction involving at least one reactant, and the artificial intelligence environment scores the set of probable reaction products based on a desired metric.

22. The system according to claim 21, further comprising an approximation module,
wherein the approximation module identifies a closest set of reactants from a set of all available reactants based on a distance in a compatible metric space.

23. The system according to claim 21, further comprising a computer-implemented agent,
wherein the computer-implemented agent operates according to a reinforcement learning process and comprises at least one actor module, and
wherein the computer-implemented agent interfaces with the artificial intelligence environment through the reinforcement learning process by providing to the artificial intelligence environment the at least reaction involving at least one reactant for the purpose of simulating a reaction and/or an action in the space of reactants.

24. The system according to claim 23, wherein the computer-implemented agent further comprises at least one critic module which is used to evaluate an output of the at least one actor module.

25. The system according to claim 22, wherein the approximation module is differentiable and is part of a computer-implemented agent, so that the approximation module may update at least one of an actor network and a critic network based on an output of the critic network by propagating a gradient through the approximation module.

26. The system according to claim 23, wherein an initial reactant is sampled randomly, is sampled by using a statistical metric, or is sampled by using a network whose output is evaluated by a critic module.

27. The system according to claim 21, wherein the at least one reaction involving at least one reactant are selected through a proto-action generated by a genetic algorithm.

28. The system according to claim 21, wherein the at least one reaction involving at least one reactant are selected by a reinforcement learning model which is trained to imitate an output of a genetic algorithm.

29. The system according to claim 27, wherein at least one actor and/or at least one critic module is/are trained based on an output of the genetic algorithm.

30. The system according to claim 21, wherein a planning method or a reinforcement learning module trained to imitate a planning method is employed to compute at least one action at every time step.

31. The system according to claim 21, wherein the artificial intelligence environment further uses at least one reaction condition in predicting the set of probable reaction products.

32. The system according to claim 21, wherein the set of probable reaction products serves as the at least one reactant of a subsequent reaction.

33. The system according to claim 21, wherein the at least one reactant comprises a tensor in a space defined by features of a set of all available reactants.

34. The system according to claim 23, wherein a critic module evaluates an output of the at least one actor module for the purpose of choosing a reactant.

35. The system according to claim 21, wherein the chemical reaction prediction module predicts at least one probable reaction product on the basis of at least one of: a rule-based algorithm, a physics-based algorithm, a quantum mechanical algorithm, a machine-learning algorithm, and a hybrid quantum machine-learning algorithm.

36. The system according to claim 21, wherein the chemical reaction prediction module predicts the set of at least one probable reaction products on the basis of an N-component transformation.

37. The system according to claim 21, wherein the scoring function module determines a reward according to at least one predicted or experimental property of the set of probable products.

38. The system according to claim 21, wherein the artificial intelligence environment uses a retrosynthesis prediction module based on at least one of: a rule-based algorithm, a quantum mechanical algorithm, a physics-based algorithm, a machine-learning algorithm and a hybrid quantum machine-learning algorithm to evaluate a synthesis process.

39. The system according to claim 37, wherein the at least one predicted property is determined by at least one of: a rule-based algorithm, a quantum mechanical algorithm, a physics-based algorithm, a machine-learning algorithms, and a hybrid quantum machine-learning algorithm.

40. A method for automated design of molecules, comprising:
using a computer-implemented agent to generate at least one reaction involving at least one reactant;
providing, by the computer-implemented agent, the at least one reaction involving at least one reactant to an artificial intelligence environment;
simulating, in the artificial intelligence environment, the at least one reaction involving at least one reactant to generate a set of at least one probable reaction product;
scoring the set of at least one probable reaction product according to a desired property; and
generating a set of optimal reaction products selected from the set of at least one probable reaction product and passing the set of optimal reaction products to the computer implemented agent to serve as a new set of reactants;
wherein the method is terminated when the set of optimal reaction products contains a desired final product.
